(19) United States
(12) Patent Application Publication
(10) Pub. No.: US 2007/0106128 A1
(43) Pub. Date: May 10, 2007

(54) Computer assisted surgery system

(76) Inventor: Stephane Lavallee, Saint Martin D'Uriage (FR)

Correspondence Address: PLEVY & HOWARD & DARCY P.C., P.O. BOX 226, Fort Washington, PA, 19034, US

(21) Appl. No.: 11/268,770

(22) Filed: Nov. 7, 2005

(51) Int. Cl. A61B 5/00

(52) U.S. Cl. 600/300

(57) ABSTRACT

The invention a computer assisted surgery system comprising a base; a telescopic mast, movable between an unextended state and an extended state, having at least first, second and third elongated portions, the third elongated portion being inserted at least partially into the second elongated portion and the second elongated portion being inserted at least partially into the first elongated portion in the unextended state, the first elongated portion being linked to the base; a monitor linked to the second elongated portion; and sensing means linked to the first elongated portion, such that when the telescopic mast is in the unextended state with the sensing means in a determined position, the system is contained in a parallelepiped of dimensions 1000 mm by 700 mm by 600 mm.

### FIELD OF THE INVENTION

The present invention relates to a computer assisted surgery system or CAS platform. In particular, the present invention relates to a CAS platform which includes a camera, a computer and a visual display.

### BACKGROUND TO THE INVENTION

Generally, in conventional computer assisted surgery systems, a measurement mark is fixed to a bone, an anatomic element of a patient or a tool used by the surgeon and its motions are followed by means of a position sensor, also known as a locating system or a tridimensional positioning system. Such a position sensor may be a set of cameras which locate the position and orientation of the measurement mark which may be formed of at least three infra-red diodes or reflective markers. Alternatively, the position of the measurement mark may be obtained by acoustic or magnetic technologies.

Images captured by the cameras are sent to a computer linked to a display screen which provides images to assist the surgeon for the preparation of a surgical operation or during a surgical operation. Usually, the cameras are fixed to the top of a mast so that images can be captured even though persons move in the operating room. Moreover, an input interface, such as a keyboard, a mouse or a foot assembly is provided so that the surgeon customizes the images displayed on the display screen.

Such computer-assisted surgery systems are usually bulky since they include the mast with the camera at its top, the screen and a holder thereof, the computer and the input interface used by the surgeon. Moreover, a specialist is required to install and dismantle such systems since many electrical and mechanical connections have to be made. This means that when a failure of the system occurs, an on-site specialist is needed to replace the failing part. Such on-site maintenance is uneasy to implement efficiently.

The computer-assisted surgery system provided by Orthosoft under the name Navitrack® Sesamoid™ includes a mast, the bottom of which is mounted to a base comprising four wheeled feet. The cameras are mounted at the top of the mast. A monitor with built-in computer is attached directly to the mast when minimal place is available. Such a computer assisted surgery system occupies a limited space.

However, a specialist is still required to install or dismantle such system since the base, the wheeled feet, the mast, the camera and the monitor have to be connected together mechanically and electrical connections have to be made at least between the monitor and the mast. Moreover, once mounted, it is not easy to move such a system over large distances since it would require a box of having large dimensions to contain it. It would also be necessary to dismantle it and mount it again at the new location, requiring the intervention of a specialist.

### SUMMARY OF THE INVENTION

One aim of the present invention is to provide a computer assisted surgery system which is mountable without the intervention of a specialist.

Another aim of the present invention is that the computer assisted surgery system is easily transportable.

To achieve these and other objects, the present invention provides a computer assisted surgery system comprising a base; a telescopic mast, movable between an unextended state and an extended state, having at least first, second and third elongated portions, the third elongated portion being inserted at least partially into the second elongated portion and the second elongated portion being inserted at least partially into the first elongated portion in the unextended state, the first elongated portion being linked to the base; a monitor; and sensing means linked to the third elongated portion, such that when the telescopic mast is in the unextended state with the sensing means in a determined position, the system is contained in a parallelepiped of dimensions 1000 mm by 700 mm by 600 mm.

According to an embodiment of the present invention, the monitor is linked to the second elongated portion.

According to an embodiment of the present invention, the second elongated portion has a first and a second end, the first end being closer to the base than the second end, the monitor being linked to the second elongated portion at the second end.

According to an embodiment of the present invention, the third elongated portion has a first and a second end, the first end being closer to the base than the second end, the sensing means being linked to the third elongated portion at the second end.

According to an embodiment of the present invention, the system comprises an arm having first and second ends, the first end being linked to the third elongated portion according to a at least one degree of liberty of rotation link, the sensing means being linked to the second end of the arm according to a at least one degree of liberty of rotation link.

According to an embodiment of the present invention, the monitor is linked to the second elongated portion according to at least a one degree of liberty of translation and one degree of liberty of rotation link.

According to an embodiment of the present invention, the system comprises at least three elongated feet, each elongated foot having first and second ends, each elongated foot being pivotally linked to the base at the first end and being linked at a pivotal wheel at the second end, the elongated feet being adapted to be folded away to be adjacent to a face of the base.

According to an embodiment of the present invention, the sensing means comprises at least a camera.

According to an embodiment of the present invention, the monitor includes a built-in computer.

According to an embodiment of the present invention, the system comprises a connection part linking the monitor to the second elongated portion, the connection part comprising a slide attached to the monitor and a guide pivotally linked to the second elongated portion, the slide being slidably mounted into the guide.

According to an embodiment of the present invention, the system comprises at least one electrical converter attached to the base and electrically linked to the monitor or/and to the sensing means by electrical conducting means at least partially located inside the telescopic mast.

According to an embodiment of the present invention, the system comprises means for moving the second elongated portion of the telescopic mast with respect to the first elongated portion of the telescopic mast.

According to an embodiment of the present invention, the system comprises a hydraulic cylinder arranged to move the second elongated portion of the telescopic mast with respect to the first elongated portion of the telescopic mast.

The present invention also provides a method for folding a computer assisted surgery system from an operation configuration to a transport configuration wherein the system is contained in a parallelepiped of dimensions 1000 mm by 700 mm by 600 mm, the computer assisted surgery system comprising a base, a telescopic mast having at least first, second and third elongated portions, the first elongated portion being linked to the base, a monitor linked to the second elongated portion, and sensing means linked to the third elongated portion, the method comprising the insertion of the third elongated portion at least partially into the second elongated portion; the insertion of the second elongated portion at least partially into the first elongated portion; and the displacement of the sensing means closer to the first elongated portion.

### BRIEF DESCRIPTION OF THE DRAWINGS

The aims, characteristics and advantages, as well as other aspects of the present invention, will be described in detail in the following description with reference to a number of examples, and with reference to the attached figures in which:

FIGS. 1 and 2 are respectively a front view and a rear view of a computer assisted surgery system according to an example of the present invention in an operation configuration;

FIG. 3 is a detailed view of a part of the computer assisted surgery system of FIG. 2;

FIGS. 4 to 6 are respectively a front view, a rear view and an underside view of the system of FIGS. 1 and 2 in a transport configuration; and

FIG. 7 is a front view of the computer assisted surgery system according to another example of the present invention in an operation configuration.

For reasons of clarity, same elements in the figures have been referenced with the same reference numbers throughout the figures.

### DETAILED DESCRIPTION OF EMBODIMENTS OF THE INVENTION

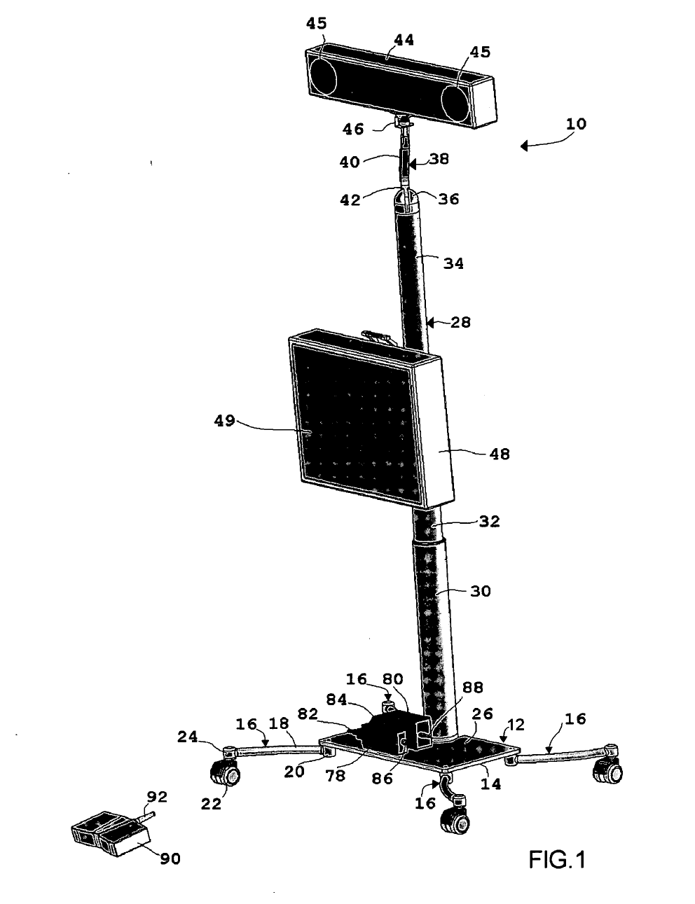

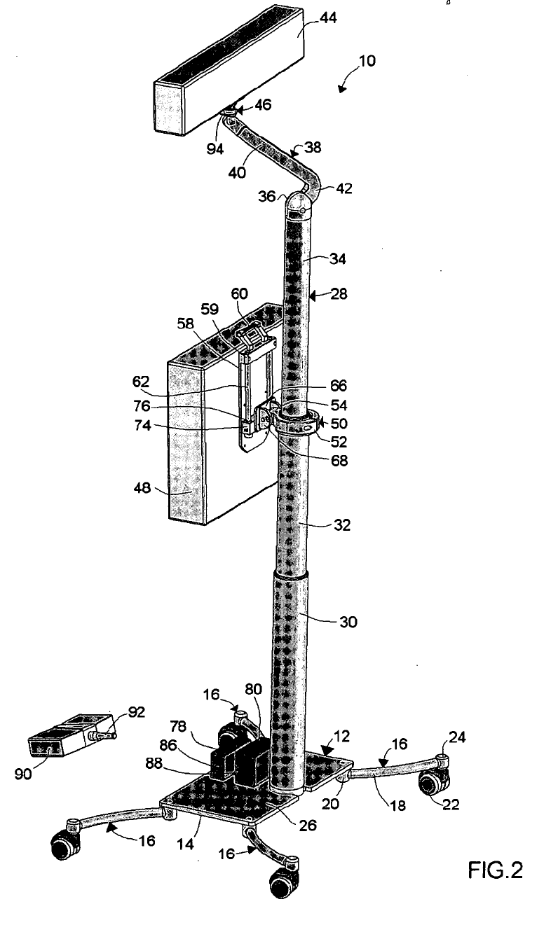

FIGS. 1 and 2 are respectively a front view and a rear view of a computer assisted surgery system 10 according to an example of the present invention in an operation configuration. System 10 includes a base 12, which, in the present example, has a generally rectangular shape. The base 12 comprises a bottom face 14 on which are mounted four pivotal feet 16. Each foot 16 has the shape of a slightly incurved rod 18. One end 20 of the rod 18 is pivotally connected to the bottom face 14 of the base 12 with respect to a vertical axis. A wheel 22 is pivotally mounted to the other end 24 of the rod 18 with respect to a vertical axis. The base 12 comprises a top face 26 opposite to the bottom face 14.

A locking mechanism, not shown, is incorporated into the feet 16 such that when each foot 16 reaches a certain angular position, it is locked into place mechanically and can only be folded back in by releasing or forcing the locking mechanism. Such a locking mechanism is realized, for example, by using a ball stop component integrated in the end 20 or rod 18 of each foot 16, where the ball falls into a recess at a certain angular position, locking the foot 16. The feet 16 can be locked in either the extended or retracted position, by placing the recesses correspondingly. Additionally, the feet 16 can be inter-connected with a pivoting linkage mechanism or a gear mechanism, not shown, such that when one foot 16 is unfolded, all feet unfold simultaneously.

A telescopic mast 28, extending in the vertical direction in the present example, comprises a lower portion 30, a middle portion 32 and an upper portion 34 and is fixed to the base 12 at an end of the lower portion 30. The three portions 30, 32, 34 are cylindrical in shape and are formed as sleeves adapted to fit into each other so that, in an unextended position, the middle portion 32 is nearly completely inserted into the lower portion 30 and the upper portion 34 is nearly completely inserted into the middle portion 32, then also into the lower portion 30. An end piece 36 is pivotally mounted at the upper end of the upper portion 34 of the mast 28 so that it is rotatable around the axis of the mast, that is to say the vertical axis in the present example. An articulated arm 38 is pivotally mounted to the end piece 36 so that it can be rotated with respect to the end piece 36 around an axis perpendicular to the axis of the mast 28, that is to say a horizontal axis in the present example. The arm 38 includes a first elongated portion 40, predominantly straight, which extends to a second portion 42 which is inclined with respect to the first portion 40 with a 90° angle. The end of portion 42 is pivotally fixed to the end piece 36. The system 10 comprises a box 44 containing one or several cameras (two cameras 45 are shown in the figures) and being generally shaped as a parallelepiped. The camera box 44 is mounted at the end of portion 40 of the arm 38 by means of a connection part 46 which is described more precisely below. The connection part 46 allows three degrees of liberty of rotation of the camera box 44 with respect to the arm 38.

A monitor 48 with built-in computer is fixed to the middle portion 32 of the telescopic mast 28 by means of a connection part 50. The monitor 48 comprises a screen 49, such as for example a touch screen, and the different common elements of a computer. For example, it can include a disk reader. As will be described below, the connection part 50 allows a displacement of the monitor 48 parallel to the middle portion 32 of the mast 28 along the axis of the mast, an inclination of the monitor 48 with respect to the middle portion 32 of the mast 28 along an axis perpendicular to the axis of the mast 28, that is to say a horizontal axis in the present example, and a swivel of the monitor 48 with respect to the axis of the mast 28.

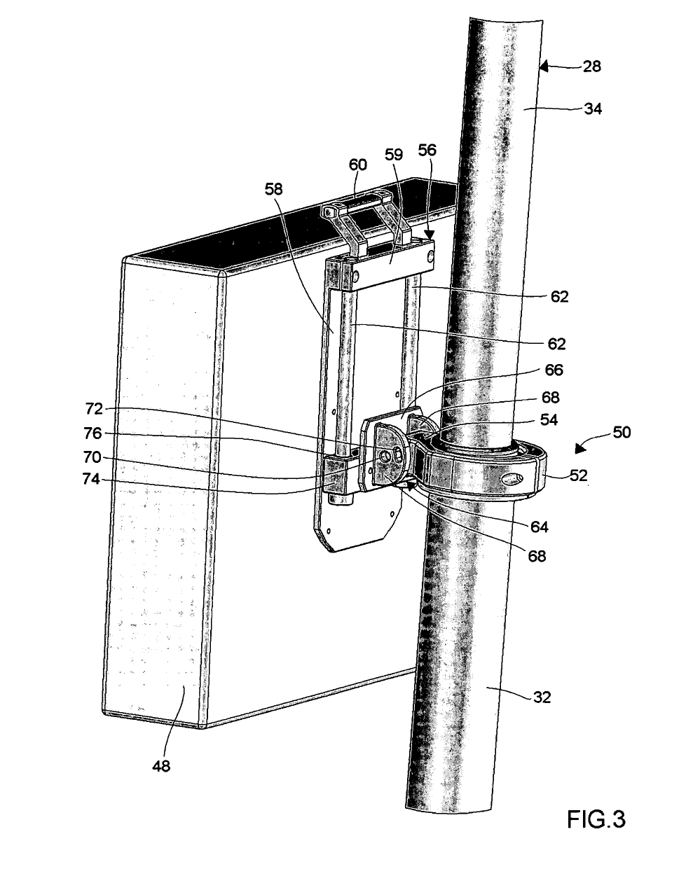

FIG. 3 shows in more detail the connection part 50 which includes a ring 52 positioned at the top of the middle portion 32 and which has a protrusion 54 extending from it. The ring 52 is pivotally mounted to the middle portion 32 of the mast 28 so that it can rotate with respect to the axis of the mast 28. The connection part 50 also includes a holder 56 fixed to the rear face of the monitor 48. The holder 56 includes a plate 58 attached to the rear face of the monitor 48, a flange 59 which is fixed to the plate 58, a handle 60 which is fixed to the plate 58 and which projects out from the top of the monitor 48 and two parallel shafts 62, fixed at one end to the plate 58 by means of the flange 59 and which extend parallel to each other and parallel to the plate 58. The connection part 50 also includes an inclinable part 64 which comprises a basis 66 with two lugs 68 extending perpendicularly to the basis 66. The lugs 68 are pivotally mounted to the protrusion 54 around a shaft 70. At least one of the lug 68 includes an opening 72 incurved around the axis of shaft 70. A pin, not shown, extends from the protrusion 54 and enters the opening 72. The cooperation of the pin and the opening 72 defines the range of allowable inclination of the inclinable part 64 with respect to the protrusion 54. The inclinable part 64 comprises a guide 74, fixed on the opposite side of the basis 66 with respect to the lugs 68. The guide 74 has two parallel cylindrical openings 76. The shafts 62 are slidably mounted into the openings 76 so that the relative position between the holder 56 and the inclinable part 64 can be modified.

The electrical supply to the cameras 45 and to the monitor 48 is provided by converters 78, 80 (or transformers) fixed to the base 12. Each converter 78, 80 is connected to a general supply network, not shown, through power supply leads 82, 84, only the ends of which are shown in the figures. The general supply network is connected to the hospital power supply with a single plug (not shown). Thus the monitor with built-in computer 48, the cameras 45, and other components can all be powered with only a single power cable. Additionally, a medial grade power supply (not shown) can be incorporated into the base 12 to provide uninterruptible power to the converters. The converter 78 is connected to the monitor 48 by means of an electrical power and communication signal cable 86, only the end of which is shown in the figures. The converter 80 is connected to the cameras 45 of the camera box 44 by means of an electrical power and communication signal cable 88, only the end of which is shown in the figures. The electrical power and communication signal cables 86, 88 to the monitor 48 and the camera box 44 can be routed either in the interior or the exterior of the hollow mast 28. For example, rings (not shown) on the exterior of the telescopic mast 28 can be used to guide the cables 86, 88 and to keep them close to the mast 28, similar to the rings on a fishing rod or pole. For example, one ring per mast portion could be used. Alternatively, the cables 86, 88 can be coiled on the inside of the mast 28, for example, around a hydraulic cylinder used to extend the mast 28 as will be described later, such that when the mast 28 is extended or retracted, the pitch of each coiled cable increases or decreases, respectively. When the mast 28 is in the fully retracted position, each cable can be completely coiled such that the coil pitch is equivalent to the thickness of the cable, thus occupying the minimal axial length.

Even though the monitor 48 may comprise a touch screen through which the surgeon inputs commands, a pedal assembly 90, actuated by the surgeon with his feet, is provided in the present example of the invention. The pedal assembly 90 includes, for example, two pedals and is connected to the monitor 48 by means of a wire 92, only the end of which is shown in the figures, which extends, for example, inside the mast 28, from the bottom of the lower portion 30 up to the monitor 48 through the connection part 50.

The computer assisted surgery system 10 is shown on FIGS. 1 to 3 in the operation configuration, that is to say ready for use. According to the embodiments of the present invention, the computer assisted surgery system 10 having the features described above can be “folded” in order to occupy a space within a parallelepiped volume with the dimensions 1000 mm by 700 mm by 600 mm, preferably 900 mm by 600 mm by 500 mm, and more preferably 800 mm by 500 mm by 350 mm. In such a configuration, the computer assisted surgery system 10 can then be placed inside a box in order to be transported. Moreover, according to embodiments of the present invention, the global weight of the computer assisted surgery system 10 and the box containing it does not exceed about 25 kg so that it can be easily transported by conventional means of transport. The portability of the computer assisted surgery system 10 in the transport configuration can be extremely useful, for example, in the following situations:
the computer assisted surgery system 10 can be taken as checked luggage on an airplane;
the computer assisted surgery system 10 can be easily folded up by a nurse and sent back to the manufacturer by standard courier, in case of malfunctioning parts for example;
the computer assisted surgery system 10 can easily be transported by sales representatives to give demonstrations in different locations; and
the computer assisted surgery system 10, in transport configuration, does not take up a lot of storage space in the hospital or in the operating room, and can easily be transferred from one department to another.

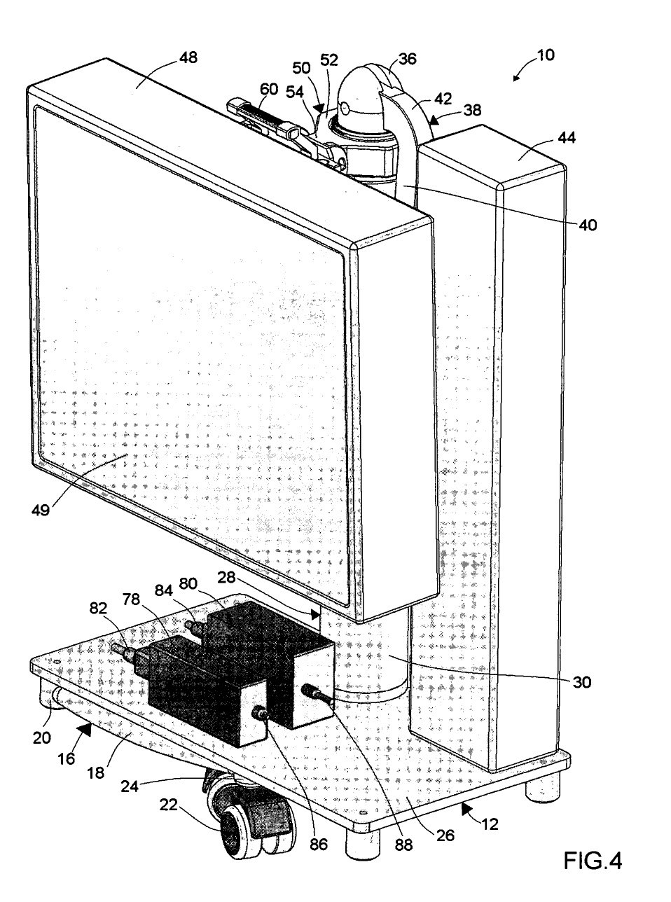

FIGS. 4 to 6 are respectively a front view, a rear view and an underside view of the system of FIGS. 1 and 2 in the transport configuration. In the transport configuration, the mast 28 is unextended. This means that the upper portion 34 and the middle portion 32 are inserted into the lower portion 30. The axial lengths of portions 30, 32, 34 are chosen such that, in the transport configuration, only the top of the middle portion 32, to which the connection part 50 is attached, remains outside the lower portion 30 and that only the end piece 36 at the top of the upper portion 34 remains outside the middle portion 32.

In the transport configuration, the monitor 48 is moved parallel to the middle portion 32 of the mast 28 so that it is placed in the lowest position possible with respect to the middle portion 32. This corresponds to the position wherein the flange 59 abuts the guide 74. In such a position, the monitor 48 rests above the converters 78, 80 at a certain distance from them. The monitor 48 is positioned vertically, such that the pedal assembly 90 is lodged between the lower portion 30 of the mast 28 and the monitor 48. For example, a holding means is provided at the back of the monitor 48 to attach the pedal assembly 90 in the transport configuration.

The arm 38 is pivoted with respect to the end piece 36 so that the straight portion 40 is brought to the closest position possible to the mast 28. The dimensions of portion 42 are determined so that, in the transport configuration, the portion 40 extends globally parallel to the mast 28, nearly in contact with it. The camera box 44 is orientated so that its longest edge extends globally parallel to the mast 28, that is to say vertically in the present example, one side of the camera box 44 lying on the top face 26 of the base 12 or being close to the top face 26 of the base 12.

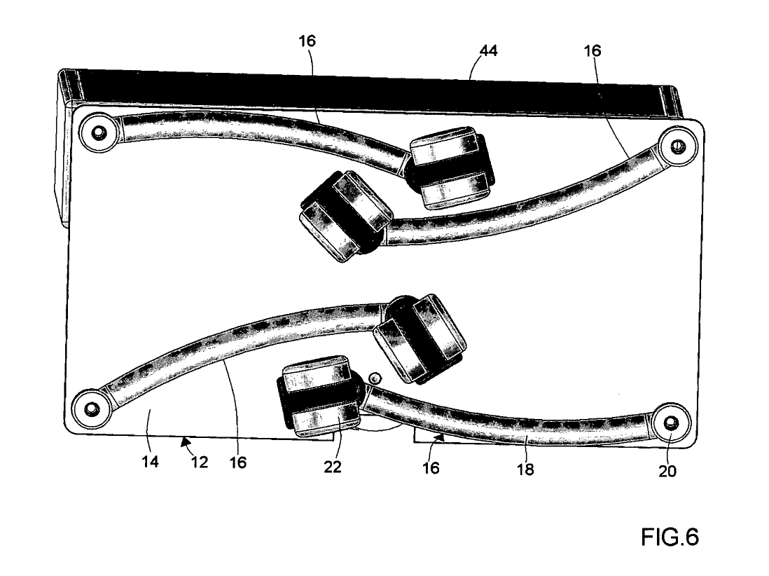

FIG. 6 represents the positions of the feet 16 of the computer assisted surgery system 10 in the transport configuration. The feet 16 are folded away under the base 12 so that no part of the feet protrudes from the underside of the base 12. The curved shape of the feet 16 makes the folding away of the feet 16 easier by preventing the feet 16 to contact to each other.

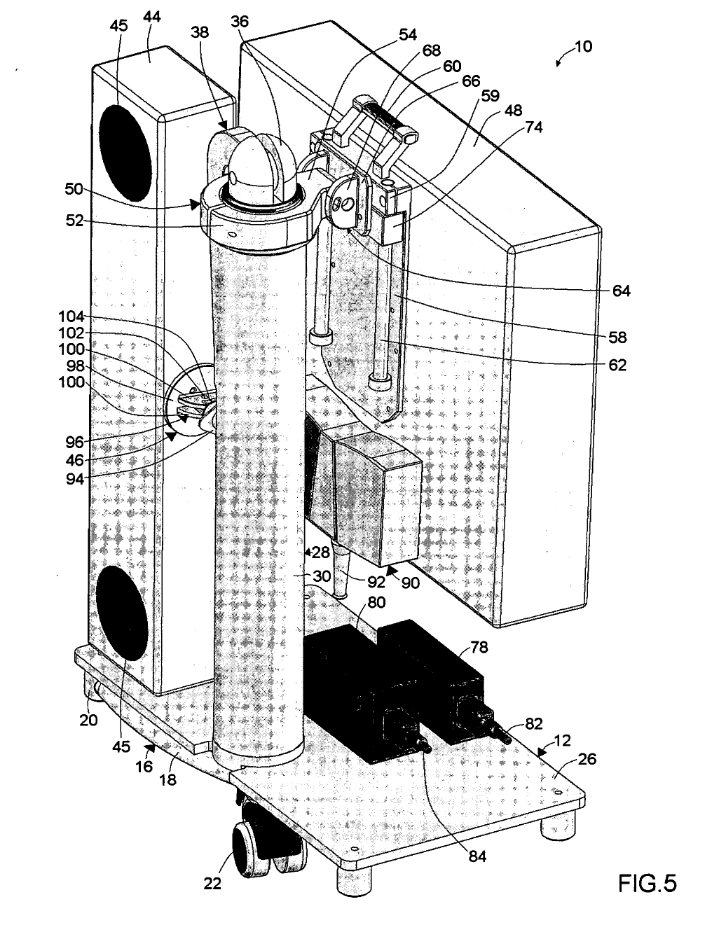

As shown on FIGS. 1 and 5, the connection part 46 includes a cylindrical part 94 which is pivotally linked to the arm 38 with respect to an axis perpendicular to the axis of the mast 28, that is to say, in the present example, vertically in the operation configuration and horizontally in the transport configuration. The connection part 46 also includes an inclinable part 96 which comprises a cylindrical basis 98 attached to the camera box 44. Two lugs 100 extend perpendicularly to the basis 98 and are pivotally mounted on the cylindrical part 94 around a shaft 102. At least one of the lug 100 includes an opening 104 incurved around the axis of shaft 102. A pin, not shown, extends from the cylindrical part 94 and enters the opening 104. The cooperation of the pin and the opening 104 defines the range of allowable inclination of the camera box 44 with respect to the cylindrical part 94.

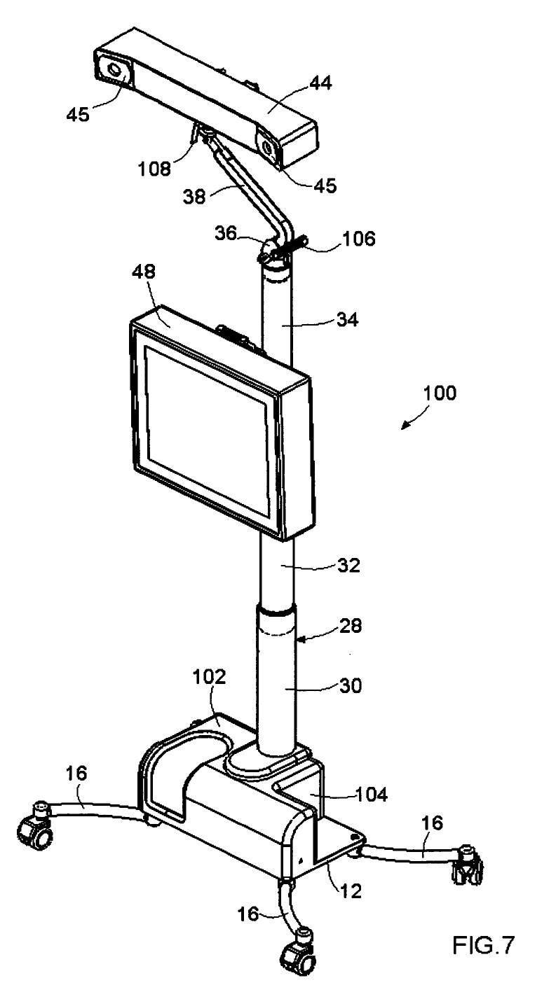

FIG. 7 is a front view of a computer assisted surgery system 100 according to another example of the present invention in the operation configuration. System 100 includes a cover 102 fixed to the base 12. The cover 102 protects the converters used for the voltage supply of the monitor 48 and the cameras 45. The cover 102 also enhances the visual aspect of the system 100. The cover 102 comprises a recess 104 in which is placed a part of the camera box 40 in the transport configuration. In this example, a supplementary handle 106 is fixed to end piece 36 to facilitate the extension of the upper portion 34 of the mast 28 with respect to the middle portion 32 and/or to facilitate the rotation of arm 38 with respect to the mast 28. Moreover, a supplementary handle 108 is fixed to camera box 44 to facilitate the positioning of the camera box 44 extension with respect to the arm 38.

An exemplary method for “unfolding” the computer assisted surgery system 10 from the transport configuration, wherein the system 10 is placed into a transport case, not shown, to the operation configuration comprises the following steps:
open the case;
unfold two feet 16;
roll out the system 10 from the case;
unfold the other two feet 16;
extend the upper portion 34 of the mast 28 from the middle portion 32;
deploy the arm 38;
extend the monitor 48 with the handle 60;
extend the middle portion 32 of the mast 28 from the lower portion 30;
deploy the pedal assembly 90 and the power cable of the system 10;
plug in the power cable; and
switch the system 10 on.

Precise positioning and orientation of the camera box 44 and of the monitor 48 is performed during or after the “unfolding” operation. Once unfolded, the feet 16 provide good stability to the system 10. The system 10 can then be moved towards any desirable location.

An exemplary method to “fold” the computer assisted surgery system 10 from the operation configuration to the transport configuration comprises the following step:
switch the system 10 off;
unplug the converters 82, 84;
insert the middle portion 32 of the mast 28 into the lower portion 30 of the mast 28;
unfold the arm 38 and orientate the camera box 44 so that it is globally parallel to the mast 28;
insert the upper portion 34 of the mast 28 into the middle portion 32, that is to say also into the lower portion 30 of the mast 28; and
fold the feet 16 away under the base 12.

In the above methods, the extension of the mast 28 is made manually or by means of an actuation mechanism, for example a hydraulic-based, a cable based or a pinion based actuation mechanism contained inside the mast 28. For example, the actuation mechanism used to lift the mast 28 is in the form of a hydraulic cylinder contained inside the hollow mast 28, similar to the hydraulic cylinder commonly used to actuate an office chair. The hydraulic cylinder comprises a cylinder and a piston placed inside the cylinder and movable with respect to the cylinder along the axis of the cylinder. The cylinder is fixed to the lower portion 30 of the mast 28 or to the base 12 and the piston is fixed to the middle portion 32, with the long axis of the cylinder parallel to the long axis of the telescopic mast 28. The cylinder contains compressed gas, and a button or foot switch (not shown) is used to release the piston such that the pressurised gas contained in the cylinder expands, moving the piston lifting the middle portion 32 of the mast 28 relative to the lower portion 30. To retract the mast 28, the button is depressed and the piston can be retracted in the cylinder by pushing down on the upper elements of the mast 28 such as the middle or the upper portions 32, 34 or handle 60, for example. Similarly, upper portion 34 can be automatically raised and lowered relative to the middle portion 32 using a hydraulic cylinder. Furthermore, said button can be connected to either one or both hydraulic cylinders using for example an ordinary mechanical cable such as those commonly found on the brakes of a bicycle.

Moreover, when the monitor 48 is positioned and oriented with respect to the middle portion 32 of the mast 28 and when the camera box 44 is positioned and oriented with respect to the upper portion 34 of the mast 28, the final chosen positions and orientations can be maintained by the friction alone between the mechanical parts. Alternatively, stop means are provided, such stop means being activated when the desired positions and orientations of the connection parts 46, 50 and of the arm 38 are achieved to block the connection parts 46, 50 and the arm 38.

The above described embodiment of the present invention has a number of advantages:

first, the computer assisted surgery system according to the above described embodiment of the present invention provides a complete system wherein the monitor with built-in computer and the cameras are fixed to a telescopic mast, hence occupying a minimal volume in transport configuration when the mast is unextended;

second, the computer assisted surgery system according to the above described embodiment of the present invention can be quickly and efficiently positioned according to the surgeon's wishes since the monitor with built-in computer and the cameras are separately positionable and orientable with respect to the mast and the system itself is mounted on wheels; and

third, the folding and unfolding methods do not involve any mechanical connection operations or electrical connection operations (except the plugging or the unplugging of the converters) and as such are implementable by a non specialist person.

According to another example of the present invention, the monitor is not attached to the telescopic mast, but is attached to a separate mast. In this case, the computer assisted surgery system has the structure described above, without the monitor, such monitor being provided separately. Indeed, in some conditions, it is preferable to place the monitor and the camera box at different locations.

It will be apparent to those skilled in the art that different connection parts 46, 50 to those previously described could be used. For example, a ball joint could be used to link the camera box 44 to the arm 38 instead of connection part 46 or to link the guide 74 to the protrusion 54 in connection part 50. Moreover, the telescopic mast 28 could have more than three portions.

Having thus described at least one illustrative embodiment of the invention, various alterations, modifications and improvements will readily occur to those skilled in the art. Such alterations, modifications and improvements are intended to be within the scope of the invention. Accordingly, the foregoing description is by way of example only and is not intended to be limiting. The invention is limited only as defined in the following claims and the equivalent thereto.

1. A computer assisted surgery system comprising:
a base;
a telescopic mast, movable between an unextended state and an extended state, having at least first, second and third elongated portions, the third elongated portion being inserted at least partially into the second elongated portion and the second elongated portion being inserted at least partially into the first elongated portion in the unextended state, the first elongated portion being linked to the base;
a monitor; and
sensing means linked to the third elongated portion,
such that when the telescopic mast is in the unextended state with the sensing means in a determined position, the system is contained in a parallelepiped of dimensions 1000 mm by 700 mm by 600 mm.

2. A system according to claim 1, wherein the monitor is linked to the second elongated portion.

3. A system according to claim 1, wherein the second elongated portion has a first and a second end, the first end being closer to the base than the second end, the monitor being linked to the second elongated portion at the second end.

4. A system according to claim 1, wherein the third elongated portion has a first and a second end, the first end being closer to the base than the second end, the sensing means being linked to the third elongated portion at the second end.

5. A system according to claim 1, comprising an arm having first and second ends, the first end being linked to the third elongated portion according to a at least one degree of liberty of rotation link, the sensing means being linked to the second end of the arm according to a at least one degree of liberty of rotation link.

6. A system according to claim 1, wherein the monitor is linked to the second elongated portion according to at least a one degree of liberty of translation and one degree of liberty of rotation link.

7. A system according to claim 1, comprising at least three elongated feet, each elongated foot having first and second ends, each elongated foot being pivotally linked to the base at the first end and being linked at a pivotal wheel at the second end, the elongated feet being adapted to be folded away to be adjacent to a face of the base.

8. A system according to claim 1, wherein the sensing means (44) comprises at least a camera.

9. A system according to claim 1, wherein the monitor includes a built-in computer.

10. A system according to claim 1, comprising a connection part linking the monitor to the second elongated portion, the connection part comprising a slide attached to the monitor and a guide pivotally linked to the second elongated portion, the slide being slidably mounted into the guide.

11. A system according to claim 1, comprising at least one electrical converter attached to the base and electrically linked to the monitor or/and to the sensing means by electrical conducting means at least partially located inside the telescopic mast.

12. A system according to claim 1, comprising means for moving the second elongated portion of the telescopic mast with respect to the first elongated portion of the telescopic mast.

13. A system according to claim 1, comprising a hydraulic cylinder arranged to move the second elongated portion of the telescopic mast with respect to the first elongated portion of the telescopic mast.

14. A method for folding a computer assisted surgery system from an operation configuration to a transport configuration wherein the system is contained in a parallelepiped of dimensions 1000 mm by 700 mm by 600 mm, the computer assisted surgery system comprising a base, a telescopic mast having at least first, second and third elongated portions, the first elongated portion being linked to the base, a monitor linked to the second elongated portion, and sensing means linked to the third elongated portion, the method comprising:
inserting the third elongated portion at least partially into the second elongated portion;
inserting the second elongated portion at least partially into the first elongated portion; and
moving the sensing means closer to the first elongated portion.
